SEQUENCE LISTING (3 sequences)

SEQ ID NO: 1 - DNA, 4748 bp, Lipomyces starkeyi
  source 1..4748: mol_type = unassigned DNA
atggctctcgcctcgaggtcgttgtccatgcccaccaaagctaccggaatcaatgacgactcacggagcagatccctgtcgcccgcgtcccgattgcggttgccctcggtccagcatccgcgccgcgcttcgtcattcagcgtattagatccgaggaggtctttctccggccctacgaccgaagctacccactggatgcgagcgcggcatgttaacgtcattctcgaactggatctgaactgcaacattatttggggtagcaagttatggacaagtgtcattgggtatgctactagactaaatcgtggtagatgatcaccaatctaacatgcgcatgcagaatcgacatacaggatctgataggaaagccaatctctgaagtactagtcggcgataaagatgtctttcaaaaggcaacagatgccatgctggagagcaacagctcctaccgcttgcggttcactactcatcgcggccatctctcacctgatcaggacgagctggagtcggttgctgtcgtcaatgatctgtcaacaccgagcagtaccgcggtcacgtcaaggacgaactccacatacgcctccacatccaattcgcgtcgcaactcgatcattagggaggaagatgcgagtaccctcgatgttacgccaaagcccacgaccgccggagtagataccggttcgtcgacgattatgcggcatttagcaattccttcgactgcaattccagcaccaacatccatgtctgcccatggagaattcgttggcgccagtccgcccaagaattctacagactcccaggaatcacatgaagacaccacggaaactacacctcccaagatattagaaatggagggtcaaggaatcatcatttttgatagacatactttggaaccttgctacgtatgcttcgttatcttcatactatgcctactcattgctaactttttatagtccatgtgggtgttgaaaccgtacgttgagccacggcgacccgtcgtcgatcttccccagatgctcgtcgactcccttgggtttggcgccgattttctcgcaaactatctctcccgactgtcccagactggcgtaacaagctttccaatgcaagatccgatcatgtgccgaatatgcgagcgacacatccagccatggtggtttgagcggcattcggagctttgtctcgtcgagcataaggccgagcgcgacgtgcagccatgtcaggacaatcttcgtgagcatagagcgtgtttgtttgccatattagagcaattagaaggacaaacgaatcacgatggagtggtcgaatatagaggaatgtcgattgtgccgcacccgttgcacaggtcaggatcgtctcagagatttccgctgtcgctctcgcatttgaagcatcttggaaagcatcggtcgccggttcgaacggtggaaatgctcctggacttgtgtgacatctcgctgttagtaaatacccccgccctgcgcgacagtaatcgaacgtccgatggagatggcggctatcaaatccgagtgcattcaccggaatccgaatctcgtttgaaacaggtcctgtcatggcacgcgccgtcgacagtcgagggtgaaggtttgatactgttatgccgagacacagagcgaattgcaaaagccaaaatcgaggctatcctgcggttaggcaacacaatcacatattccgagaaaattcagcgagaacttagtgtcctcgtgaacgagactattgaagagactgtctcccacataatgcagcaacaagtcgacgaagctgatcagagcggtgaagaagatatcgcgtctgacgtcgaagatcttggattcttttcgggatatatgtcgcagcagtttagagacgagtatagtgacgatcgtgtaccctcgcccagtccaggcggatacaactctagcacgatgtctgccacgtccggatcctcgccacgagaaacactgacgccaaaatcgctattcgcgtcgctgagcagaccgaagtctcgcagattgaccatccaggacgaagaaggaatggaaagtgacagcagtcgttcatcactattctatctgcgtgagaaggccgattctcctgtcagtgagcccgaaatgccaagtcgcaatatccgggcccgaaaaagcagtgcaagtttattctttggctcgccgcatcgacagaagtcgccgtcaatctctttctcgaagtcaccgttattgaatcagaagctcaagttagcgtccatggcggataacgggtcgagcacttatagcagcaacacgagcagcactactgcccctacagagagtatcaataatatctcattcactccgctcacatctccgcttctattcccgattgattcgtatccagagcttgggcgccatcatagacgcaaatcttctgcggcgtcagatttctcgaaagctcctgtgtcaccgctgttgtcgtcagtcactccgaccattcgtccggcgcagccaagcatcaaggactttgaagtcatcaagcctatcagtcgcggtgcatttggcagtgtctatctgaccaagaagaaaagtactggcgactatttcgctataaaggttctcaaaaagtcagatatgatcgcgaagaatcaagtcacgaacgtcaaagcggagcgtgccatcatgatggcgcaggctgagagtccatttgtcgcgaaattgttcttcacgttccaaagtaaggagtacttgtatctcgtgatggaatatatgaacggtggtgattgtgccgcgctattgaaggcgtttggaggtggattcccggaggattgggcgaagaagtatgttgcggaggtggtgcttgggattgagaatttgcatgagcagggtattgtacatcggtatgtttattcctacggcttccgcaaatacaagctaacaaatgatagtgacttgaaaccggacaacctgctgatcgacaaggacggccacttgaagctcactgattttggtctttcgcgaatgggtttggtcgggcggcaagcccgtgctcgaaacgcgagcacaatcgatccgcctgatatttttcaacagggccagttcggcggccggtcaatgtcaatttccgcccaaccgggatcgcttgatacgccgctgtatgtcgacaacagtttgggtcttggcatatcacctggttcaacgccggtcatgcagccaactgattcgggggcgctgttaaatgtaccggggtatttctccctgaacaaaagcgcgctgagcgaatcatattcaaaatcgctatcgctatctaactccgccccgagaaaaggcgaatcgataaactctatgctgagttcgttcagtctaaccaatcctatgtccgcggctctccggcagtcgagcaaagagaaggaagacgatatacagagcacggcaagctcagtctccagcgaagttagcggcgtaaacggtctcggtatccaccggatgaactcgcattacaactctgcctcgggctctatttccggctccggctccggttctggctcgacaacggcttcgtcatcgatgccgccgccaccgactccagcaccgcagaatatggcgttgttcgatcctgcgaatgatggtaagaatttcgtcggcacgccggattaccttgcacccgagacgattatgggtacgggtcaggatgaaatgagcgattggtggtcactgggatgcattgtgtttgagtttctgtgcggttatcctccgttccatgccgacacaccgaatgaggtttttgagaatattcttgctcgaaaaatcgcgtggccgagtcccgaagaaatgcagtctctgtctctgtcgcctgaagctattgatctgatcgagaagcttatttgcacggaccctgaaaagcgtttgggtgcagagacgtggggccaggttaagcaacatccgttttttgaaggtatcacttgggaaaccttgtgtgatgacgaagcgccgtttattccgcagccgcttcacgaggaggatacagcttattttgacgatcgcggggcaacgttgcagacgttccctgaagaagagaatgaagattctgatgcgaagaaagacggcaaaggtcacgatcacctggcgactctagattcaagacggtcgagcgagcgcgagtcgagttccgagtccaatagcaggaaagattcattcggaagtgcggcgagcgggactaccgcgccgacaagttcgagcacacggtcaacgaaattattaccgttacatattccgcatcatttccggggtcgtcgtatgcgtcgtttgagtgaacctgtaccggcggatgattttgggtcgttctcgttcaagaacttgaacgtgctggagaaagccaataaggacgtcattcggcggttgaaatctgagcatatggaacagatgaaatcgcctgtctcgcccgcgcagccgtctgggcttatcgagccgtcacctgtgaagcctcggaatgcgtcagttgggtctcttggtgcagccatctcattgaactacggcaagcggccgggatccccgtccagtagttcttcgagtagttcgagtttattccgcaacgtctcaccgagcaggaaccttgcgcatctctcaatcccgtcgtccccgctgaccacgtccgtcccgattagtgcgccgtacgatatcgacgacgatcgagagcagccgtcgaggtcgacctcagtcattggtagtcagcccggctcgccaacaagctcacgaaaagtgtcgtattcatcgtttaaataa

SEQ ID NO: 2 - protein, 1530 aa, Lipomyces starkeyi
MALASRSLSMPTKATGINDDSRSRSLSPASRLRLPSVQHPRRASSFSVLDPRRSFSGPTTEATHWMRARHVNVILELDLNCNIIWGSKLWTSVIGIDIQDLIGKPISEVLVGDKDVFQKATDAMLESNSSYRLRFTTHRGHLSPDQDELESVAVVNDLSTPSSTAVTSRTNSTYASTSNSRRNSIIREEDASTLDVTPKPTTAGVDTGSSTIMRHLAIPSTAIPAPTSMSAHGEFVGASPPKNSTDSQESHEDTTETTPPKILEMEGQGIIIFDRHTLEPCYSMWVLKPYVEPRRPVVDLPQMLVDSLGFGADFLANYLSRLSQTGVTSFPMQDPIMCRICERHIQPWWFERHSELCLVEHKAERDVQPCQDNLREHRACLFAILEQLEGQTNHDGVVEYRGMSIVPHPLHRSGSSQRFPLSLSHLKHLGKHRSPVRTVEMLLDLCDISLLVNTPALRDSNRTSDGDGGYQIRVHSPESESRLKQVLSWHAPSTVEGEGLILLCRDTERIAKAKIEAILRLGNTITYSEKIQRELSVLVNETIEETVSHIMQQQVDEADQSGEEDIASDVEDLGFFSGYMSQQFRDEYSDDRVPSPSPGGYNSSTMSATSGSSPRETLTPKSLFASLSRPKSRRLTIQDEEGMESDSSRSSLFYLREKADSPVSEPEMPSRNIRARKSSASLFFGSPHRQKSPSISFSKSPLLNQKLKLASMADNGSSTYSSNTSSTTAPTESINNISFTPLTSPLLFPIDSYPELGRHHRRKSSAASDFSKAPVSPLLSSVTPTIRPAQPSIKDFEVIKPISRGAFGSVYLTKKKSTGDYFAIKVLKKSDMIAKNQVTNVKAERAIMMAQAESPFVAKLFFTFQSKEYLYLVMEYMNGGDCAALLKAFGGGFPEDWAKKYVAEVVLGIENLHEQGIVHRDLKPDNLLIDKDGHLKLTDFGLSRMGLVGRQARARNASTIDPPDIFQQGQFGGRSMSISAQPGSLDTPLYVDNSLGLGISPGSTPVMQPTDSGALLNVPGYFSLNKSALSESYSKSLSLSNSAPRKGESINSMLSSFSLTNPMSAALRQSSKEKEDDIQSTASSVSSEVSGVNGLGIHRMNSHYNSASGSISGSGSGSGSTTASSSMPPPPTPAPQNMALFDPANDGKNFVGTPDYLAPETIMGTGQDEMSDWWSLGCIVFEFLCGYPPFHADTPNEVFENILARKIAWPSPEEMQSLSLSPEAIDLIEKLICTDPEKRLGAETWGQVKQHPFFEGITWETLCDDEAPFIPQPLHEEDTAYFDDRGATLQTFPEEENEDSDAKKDGKGHDHLATLDSRRSSERESSSESNSRKDSFGSAASGTTAPTSSSTRSTKLLPLHIPHHFRGRRMRRLSEPVPADDFGSFSFKNLNVLEKANKDVIRRLKSEHMEQMKSPVSPAQPSGLIEPSPVKPRNASVGSLGAAISLNYGKRPGSPSSSSSSSSSLFRNVSPSRNLAHLSIPSSPLTTSVPISAPYDIDDDREQPSRSTSVIGSQPGSPTSSRKVSYSSFK

SEQ ID NO: 3 - DNA, 4593 bp, Lipomyces starkeyi
  source 1..4593: mol_type = unassigned DNA
atggctctcgcctcgaggtcgttgtccatgcccaccaaagctaccggaatcaatgacgactcacggagcagatccctgtcgcccgcgtcccgattgcggttgccctcggtccagcatccgcgccgcgcttcgtcattcagcgtattagatccgaggaggtctttctccggccctacgaccgaagctacccactggatgcgagcgcggcatgttaacgtcattctcgaactggatctgaactgcaacattatttggggtagcaagttatggacaagtgtcattggaatcgacatacaggatctgataggaaagccaatctctgaagtactagtcggcgataaagatgtctttcaaaaggcaacagatgccatgctggagagcaacagctcctaccgcttgcggttcactactcatcgcggccatctctcacctgatcaggacgagctggagtcggttgctgtcgtcaatgatctgtcaacaccgagcagtaccgcggtcacgtcaaggacgaactccacatacgcctccacatccaattcgcgtcgcaactcgatcattagggaggaagatgcgagtaccctcgatgttacgccaaagcccacgaccgccggagtagataccggttcgtcgacgattatgcggcatttagcaattccttcgactgcaattccagcaccaacatccatgtctgcccatggagaattcgttggcgccagtccgcccaagaattctacagactcccaggaatcacatgaagacaccacggaaactacacctcccaagatattagaaatggagggtcaaggaatcatcatttttgatagacatactttggaaccttgctactccatgtgggtgttgaaaccgtacgttgagccacggcgacccgtcgtcgatcttccccagatgctcgtcgactcccttgggtttggcgccgattttctcgcaaactatctctcccgactgtcccagactggcgtaacaagctttccaatgcaagatccgatcatgtgccgaatatgcgagcgacacatccagccatggtggtttgagcggcattcggagctttgtctcgtcgagcataaggccgagcgcgacgtgcagccatgtcaggacaatcttcgtgagcatagagcgtgtttgtttgccatattagagcaattagaaggacaaacgaatcacgatggagtggtcgaatatagaggaatgtcgattgtgccgcacccgttgcacaggtcaggatcgtctcagagatttccgctgtcgctctcgcatttgaagcatcttggaaagcatcggtcgccggttcgaacggtggaaatgctcctggacttgtgtgacatctcgctgttagtaaatacccccgccctgcgcgacagtaatcgaacgtccgatggagatggcggctatcaaatccgagtgcattcaccggaatccgaatctcgtttgaaacaggtcctgtcatggcacgcgccgtcgacagtcgagggtgaaggtttgatactgttatgccgagacacagagcgaattgcaaaagccaaaatcgaggctatcctgcggttaggcaacacaatcacatattccgagaaaattcagcgagaacttagtgtcctcgtgaacgagactattgaagagactgtctcccacataatgcagcaacaagtcgacgaagctgatcagagcggtgaagaagatatcgcgtctgacgtcgaagatcttggattcttttcgggatatatgtcgcagcagtttagagacgagtatagtgacgatcgtgtaccctcgcccagtccaggcggatacaactctagcacgatgtctgccacgtccggatcctcgccacgagaaacactgacgccaaaatcgctattcgcgtcgctgagcagaccgaagtctcgcagattgaccatccaggacgaagaaggaatggaaagtgacagcagtcgttcatcactattctatctgcgtgagaaggccgattctcctgtcagtgagcccgaaatgccaagtcgcaatatccgggcccgaaaaagcagtgcaagtttattctttggctcgccgcatcgacagaagtcgccgtcaatctctttctcgaagtcaccgttattgaatcagaagctcaagttagcgtccatggcggataacgggtcgagcacttatagcagcaacacgagcagcactactgcccctacagagagtatcaataatatctcattcactccgctcacatctccgcttctattcccgattgattcgtatccagagcttgggcgccatcatagacgcaaatcttctgcggcgtcagatttctcgaaagctcctgtgtcaccgctgttgtcgtcagtcactccgaccattcgtccggcgcagccaagcatcaaggactttgaagtcatcaagcctatcagtcgcggtgcatttggcagtgtctatctgaccaagaagaaaagtactggcgactatttcgctataaaggttctcaaaaagtcagatatgatcgcgaagaatcaagtcacgaacgtcaaagcggagcgtgccatcatgatggcgcaggctgagagtccatttgtcgcgaaattgttcttcacgttccaaagtaaggagtacttgtatctcgtgatggaatatatgaacggtggtgattgtgccgcgctattgaaggcgtttggaggtggattcccggaggattgggcgaagaagtatgttgcggaggtggtgcttgggattgagaatttgcatgagcagggtattgtacatcgtgacttgaaaccggacaacctgctgatcgacaaggacggccacttgaagctcactgattttggtctttcgcgaatgggtttggtcgggcggcaagcccgtgctcgaaacgcgagcacaatcgatccgcctgatatttttcaacagggccagttcggcggccggtcaatgtcaatttccgcccaaccgggatcgcttgatacgccgctgtatgtcgacaacagtttgggtcttggcatatcacctggttcaacgccggtcatgcagccaactgattcgggggcgctgttaaatgtaccggggtatttctccctgaacaaaagcgcgctgagcgaatcatattcaaaatcgctatcgctatctaactccgccccgagaaaaggcgaatcgataaactctatgctgagttcgttcagtctaaccaatcctatgtccgcggctctccggcagtcgagcaaagagaaggaagacgatatacagagcacggcaagctcagtctccagcgaagttagcggcgtaaacggtctcggtatccaccggatgaactcgcattacaactctgcctcgggctctatttccggctccggctccggttctggctcgacaacggcttcgtcatcgatgccgccgccaccgactccagcaccgcagaatatggcgttgttcgatcctgcgaatgatggtaagaatttcgtcggcacgccggattaccttgcacccgagacgattatgggtacgggtcaggatgaaatgagcgattggtggtcactgggatgcattgtgtttgagtttctgtgcggttatcctccgttccatgccgacacaccgaatgaggtttttgagaatattcttgctcgaaaaatcgcgtggccgagtcccgaagaaatgcagtctctgtctctgtcgcctgaagctattgatctgatcgagaagcttatttgcacggaccctgaaaagcgtttgggtgcagagacgtggggccaggttaagcaacatccgttttttgaaggtatcacttgggaaaccttgtgtgatgacgaagcgccgtttattccgcagccgcttcacgaggaggatacagcttattttgacgatcgcggggcaacgttgcagacgttccctgaagaagagaatgaagattctgatgcgaagaaagacggcaaaggtcacgatcacctggcgactctagattcaagacggtcgagcgagcgcgagtcgagttccgagtccaatagcaggaaagattcattcggaagtgcggcgagcgggactaccgcgccgacaagttcgagcacacggtcaacgaaattattaccgttacatattccgcatcatttccggggtcgtcgtatgcgtcgtttgagtgaacctgtaccggcggatgattttgggtcgttctcgttcaagaacttgaacgtgctggagaaagccaataaggacgtcattcggcggttgaaatctgagcatatggaacagatgaaatcgcctgtctcgcccgcgcagccgtctgggcttatcgagccgtcacctgtgaagcctcggaatgcgtcagttgggtctcttggtgcagccatctcattgaactacggcaagcggccgggatccccgtccagtagttcttcgagtagttcgagtttattccgcaacgtctcaccgagcaggaaccttgcgcatctctcaatcccgtcgtccccgctgaccacgtccgtcccgattagtgcgccgtacgatatcgacgacgatcgagagcagccgtcgaggtcgacctcagtcattggtagtcagcccggctcgccaacaagctcacgaaaagtgtcgtattcatcgtttaaataa

(19) United States
(12) Patent Application Publication
(10) Pub. No.: US 2025/0163439 A1
(43) Pub. Date: May 22, 2025

(54) LIPID PRODUCTION CONTROL FACTOR FOR OLEAGINOUS YEAST

(71) Applicant: FUJI OIL HOLDINGS INC., Izumisano-shi, Osaka (JP)

(72) Inventors: Hiroaki TAKAKU, Niigata-shi, Niigata (JP); Rikako SATO, Niigata-shi, Niigata (JP); Harutake YAMAZAKI, Niigata-shi, Niigata (JP); Hideo ARAKI, Tsukubamirai-shi, Ibaraki (JP)

(21) Appl. No.: 18/839,920

(22) PCT Filed: Feb. 1, 2023
(86) PCT No.: PCT/JP2023/003246

§ 371 Date: Aug. 20, 2024

(30) Foreign Application Priority Data
Feb. 21, 2022 (JP) 2022-024585

(51) Int. Cl. C12N 15/81; C07K 14/39; C12P 7/64

(52) U.S. Cl. CPC C12N 15/81; C07K 14/39; C12P 7/64

(57) ABSTRACT

To provide an oil and/or fat production control factor in a yeast. A yeast 110315 gene as an oil and/or fat production control factor.

### TECHNICAL FIELD

The present invention relates to an oil and/or fat production control factor of an oleaginous yeast, and the like.

### BACKGROUND ART

Oils and/or fats are closely related to our life, and their uses are mainly classified into food uses and industrial uses. The food uses include food processed oils and/or fats, which are used as salad oils in cooking, and are also processed into margarine, dressings, and the like from oil and/or fat raw materials. The industrial uses include oils and/or fats which are used as-is as fuel or lubricants, and also used as oil and/or fat chemical products such as shampoo, conditioner, or cosmetics after chemical conversion.

Raw materials of three major oils and/or fats (soybean oil, rapeseed oil, and palm oil) are mainly vegetable oils obtained by pressing seeds such as rapeseed or soybean, and fruits of palm, olive, or the like. Outside Japan, oils and/or fats can be supplied by cultivating oil plants as raw materials for oils and/or fats. On the other hand, in Japan, it is necessary to develop an oil and/or fat production system suitable for Japan to enhance the self-sufficiency rate for edible oils and/or fats.

At present, as a production method of oils and/or fats, production of oils and/or fats using microorganisms is attracting attention instead of production of oils and/or fats from oil plants. Among them, an oleaginous yeast such as a yeast belonging to the genus Lipomyces has a high oil and/or fat accumulation ability.

### CITATION LIST

### Patent Document

Patent Document 1: JP 2019-146543 A
Patent Document 2: JP 2021-132601 A

### Non-Patent Document

Non-Patent Document 1: H. Yamazaki, S. Kobayashi, S. Ebina, S. Abe, S. Ara, Y. Shida, W. Ogasawara, K. Yaoi, H. Araki & H. Takaku: Appl. Microbiol. Biotechnol., 103, 6297 (2019).
Non-Patent Document 2: Takaku, H.; Ebina, S.; Kasuga, K.; Sato, R.; Ara, S.; Kazama, H.; Matsuzawa, T.; Yaoi, K.; Araki, H.; Shida, Y. et al. J. Biosci. Bioeng. 2021, 131, 613-621

### SUMMARY OF INVENTION

### Technical Problem

In Patent Document 1, Non-Patent Document 1, and Non-Patent Document 2, it is reported that yeasts belonging to the genus Lipomyces were subjected to mutation treatment, and a yeast with higher oil and/or fat production ability was screened. However, an oil and/or fat production control factor has not been clarified. In Patent Document 2, three factors controlling the productivity of oils and/or fats have been clarified by utilizing genetic analysis data of a mutant strain. The metabolic pathways of oils and/or fats are complex and long, and thus it is conceivable that a plurality of factors controlling the production of oils and/or fats are involved in these pathways. If a new factor for controlling the productivity of oils and/or fats is further clarified, the degree of freedom for regulating the oil and/or fat production ability of a yeast increases, and by stacking such factors, it becomes possible to produce a yeast with higher oil and/or fat production ability.

Accordingly, the present invention is directed to providing an oil and/or fat production control factor in a yeast.

### Solution to Problem

As a result of intensive studies, the present inventors have found that a yeast 110315 gene is an oil and/or fat production control factor and that the oil and/or fat production ability can be regulated by regulating expression and/or function of this gene. As a result of further studies based on these findings, the present inventors have completed the present invention. That is, the present invention includes the following aspects.

Item 1. A production method of a yeast with regulated oil and/or fat production ability, the production method including regulating expression and/or function of a yeast 110315 gene in a yeast.

Item 2. The production method according to item 1, including reducing the expression and/or function of the yeast 110315 gene, and producing a yeast with improved oil and/or fat production ability.

Item 3. The production method according to item 1 or 2, including reducing the expression of the yeast 110315 gene, and producing a yeast with improved oil and/or fat production ability.

Item 4. The production method according to any of items 1 to 3, wherein the yeast is an oleaginous yeast.

Item 5. The production method according to any of items 1 to 4, wherein the yeast is a yeast belonging to the genus Lipomyces.

Item 6. A reagent for production of a yeast with regulated oil and/or fat production ability, the reagent including at least one selected from the group consisting of an expression regulator and a function regulator of a yeast 110315 gene.

Item 7. The reagent for production according to item 6, wherein the reagent includes at least one selected from the group consisting of an expression inhibitor and a function inhibitor of the yeast 110315 gene and is for use in the production of a yeast with improved oil and/or fat production ability.

Item 8. The reagent for production according to item 6 or 7, wherein the reagent includes an expression inhibitor of the yeast 110315 gene and is for use in the production of a yeast with improved oil and/or fat production ability.

Item 9. An oil and/or fat producing yeast, which is in a state where expression of a yeast 110315 gene is reduced.

Item 10. A composition for producing an oil and/or fat, the composition containing the oil and/or fat producing yeast according to item 9.

Item 11. A production method of an oil and/or fat, the method including recovering an oil and/or fat from at least one selected from the group consisting of a culture of the oil and/or fat producing yeast according to item 9 and the composition for producing an oil and/or fat according to item 10.

### Advantageous Effects of Invention

According to the present invention, it is possible to provide a production method of a yeast with regulated oil and/or fat production ability, a reagent for production of a yeast with regulated oil and/or fat production ability, an oil and/or fat producing yeast, a composition for producing an oil and/or fat, a production method of an oil and/or fat, and the like, based on a newly found oil and/or fat production control factor.

### BRIEF DESCRIPTION OF DRAWINGS

FIG. 1-1 indicates analysis results of the effect of deletion of Transcript Id:110315 on growth and oil and/or fat productivity (Test Example 2). Culture was performed in an S medium containing 5% of glucose at 30° C. and 160 rpm for 5 days. Indicated are: (A) cell concentration, (B) residual glucose amount, (C) TAG amount per medium, and (D) TAG amount per cell. The TAG amount per cell was calculated from a calculation formula of TAG amount per medium (g/L)/cell concentration (×10⁸ cells mL). Results each represent the mean of three independent results, and error bars each indicate the standard error.

FIG. 1-2 indicates analysis results of the effect of deletion of Transcript Id:110315 on growth and oil and/or fat productivity (Test Example 2). Indicated is: (E) observation images of oil and/or fat production amounts by a differential interference microscope.

FIG. 2-1 indicates analysis results of the effect of combination of 115694 and mutant substitution or deletion of Transcript Id:110315 on growth and oil and/or fat productivity of a strain (Test Example 2). Culture was performed in an S medium containing 7% of glucose at 30° C. and 160 rpm for 3 days. Indicated are: (A) cell concentration, (B) residual glucose amount, (C) TAG amount per medium, and (D) TAG amount per cell. The TAG amount per cell was calculated from a calculation formula of TAG amount per medium (g/L)/cell concentration (×10⁸ cells mL). Results each represent the mean of three independent results, and error bars each indicate the standard error.

FIG. 2-2 indicates analysis results of the effect of combination of 115694 and mutant substitution or deletion of Transcript Id:110315 on growth and oil and/or fat productivity of a strain (Test Example 2). Indicated is: (E) observation images of oil and/or fat production amounts by a differential interference microscope.

### DESCRIPTION OF EMBODIMENTS

In the present specification, the expressions “contain” and “include” include the concepts of “containing”, “including”, “consisting essentially of” and “consisting only of”.

### 1. Definition

In the present specification, the term “yeast . . . gene” means genes identified by a database of genes and the like (corresponding to Transcript Id numbers (“ . . . (multi-digit number)” in the term) in Lipomyces starkeyi in https://mycocosm.jgi.doe.gov/Lipst1_1/Lipst1_1.home.html) and the orthologous gene of the gene.

In the present specification, the expressions “contain” and “include” include the concepts of “containing”, “including”, “consisting essentially of” and “consisting only of”.

In the present specification, an “identity” of amino acid sequences refers to the extent to which two or more comparable amino acid sequences match each other in amino acid sequence. Thus, the higher the conformance of certain two amino acid sequences, the higher the identity or similarity of those sequences. The level of amino acid sequence identity is determined, for example, by using FASTA, which is a sequence analysis tool, and using default parameters. Alternatively, it can be determined by using an algorithm BLAST by Karlin and Altschul (Karlin S, Altschul SF. “Methods for assessing the statistical significance of molecular sequence features by using general scoring schemes” Proc Natl Acad Sci USA. 87: 2264-2268 (1990); Karlin S, Altschul SF. “Applications and statistics for multiple high-scoring segments in molecular sequences.” Proc Natl Acad Sci USA. 90: 5873-7 (1993)). A program called BLASTX based on such a BLAST algorithm has been developed. Specific techniques for these analysis methods are known, and reference only needs to be made to the website (http://www.ncbi.nlm.nih.gov/) of the National Center of Biotechnology Information (NCBI). “Identity” of a base sequence is also defined pursuant to the above description. In the present specification, “conservative substitution” means that an amino acid residue is substituted with an amino acid residue having a similar side chain. For example, substitution between amino acid residues having a basic side chain, such as lysine, arginine, or histidine, corresponds to conservative substitution. Similarly, substitution between amino acid residues having an acidic side chain, such as aspartic acid or glutamic acid; amino acid residues having an uncharged polar side chain, such as glycine, asparagine, glutamine, serine, threonine, tyrosine, or cysteine; amino acid residues having a nonpolar side chain, such as alanine, valine, leucine, isoleucine, proline, phenylalanine, methionine, or tryptophan; amino acid residues having a β-branched side chain, such as threonine, valine, or isoleucine; and amino acid residues having an aromatic side chain, such as tyrosine, phenylalanine, tryptophan, or histidine also corresponds to conservative substitution.

In the present specification, a polynucleotide such as DNA or RNA may be subjected to known chemical modification as exemplified below. To prevent degradation by a hydrolytic enzyme such as nuclease, the phosphoric acid residue (phosphate) of each nucleotide can be substituted with a chemically modified phosphoric acid residue such as phosphorothioate (PS), methylphosphonate, or phosphorodithioate. The hydroxyl group at the 2-position of the sugar (ribose) of each ribonucleotide may be substituted with —OR (R represents, for example, CH3(2′-O-Me), CH2CH2OCH3(2′-O-MOE), CH2CH2NHC(NH)NH2, CH2CONHCH3, CH2CH2CN, or the like). Furthermore, a base moiety (pyrimidine, purine) may be chemically modified, and for example, introduction of a methyl group or a cationic functional group into the 5-position of a pyrimidine base, substitution of a carbonyl group at the 2-position with thiocarbonyl, or the like is exemplified. Further examples of the chemical modification include, but are not limited to, those in which a phosphoric acid moiety or a hydroxyl moiety is modified with biotin, an amino group, a lower alkylamine group, an acetyl group, or the like. BNA (LNA) or the like in which the 2′ oxygen and the 4′ carbon of a sugar moiety of a nucleotide are crosslinked to fix the conformation of the sugar moiety to the N-type can also be preferably used.

### 2. Production Method of Yeast with Regulated Oil and/or Fat Production Ability

In one aspect, the present invention relates to a production method of a yeast with regulated oil and/or fat production ability, the method including regulating expression and/or function of the yeast 110315 gene in a yeast (in the present specification, also referred to as “the production method of a yeast according to the present invention”). This will be described below.

The yeast is not particularly limited, as long as it is a yeast capable of producing an oil and/or fat. The yeast is more preferably an oleaginous yeast. The oleaginous yeast is not particularly limited, as long as it is a yeast having a high oil and/or fat accumulation property. For example, the oleaginous yeast is a yeast capable of achieving a high oil and/or fat content, for example, an oil and/or fat content of 20% (w/w) or more, preferably 30% (w/w) or more, more preferably 40% (w/w) or more, even more preferably 50% (w/w) or more, and still even more preferably 60% (w/w) or more. Specific examples of the oleaginous yeast include yeasts belonging to the genus Rhodosporodium, such as Rhodosporodium toruloides; yeasts belonging to the genus Lipomyces, such as Lipomyces starkeyi; yeasts belonging to the genus Cryptococcus, such as Cryptococcus albidus; yeasts belonging to the genus Rhizopus, such as Rhizopus arrhizua; and yeasts belonging to the genus Yarrowia, such as Yarrowia lipolytica. Among them, yeasts belonging to the genus Rhodosporodium, yeasts belonging to the genus Lipomyces, yeasts belonging to the genus Cryptococcus, and the like are preferable, yeasts belonging to the genus Lipomyces and the like are more preferable, and Lipomyces starkeyi and the like are even more preferable.

The base sequence and the amino acid sequence of the yeast 110315 gene are known, or can be easily determined based on the base sequence and the amino acid sequence of the known yeast 110315 gene (for example, by identity analysis with them). As an example, the amino acid sequence represented by SEQ ID NO: 2 is exemplified as the amino acid sequence of the yeast 110315 gene of Lipomyces starkeyi and the base sequence represented by SEQ ID NO: 3 is exemplified as the base sequence encoding the protein of the yeast 110315 gene of Lipomyces starkeyi.

In the present specification, the yeast 110315 gene is also referred to as a “subject gene”.

The subject gene also includes a mutant that can occur in nature. The subject gene may have base mutation such as substitution, deletion, addition, or insertion as long as the properties of the protein to be encoded are not significantly impaired. The mutation is preferably mutation causing no amino acid substitution or mutation causing conservative amino acid substitution in a protein translated from the mRNA.

The subject gene is, for example, a gene in which the amino acid sequence of a protein encoded thereby has, for example, 95% or more, preferably 98% or more, more preferably 99% or more of identity with the amino acid sequence of a protein encoded by a wild-type subject gene of an animal of the same species. In addition, the subject gene is, for example, a gene in which the amino acid sequence of a protein encoded thereby is the same as the amino acid sequence of a protein encoded by a wild-type subject gene of an animal of the same species, or an amino acid sequence in which one or more (e.g., 2 to 10, preferably 2 to 5, more preferably 2 to 3, even more preferably 2) amino acids are substituted, deleted, added, or inserted, for the amino acid sequence.

“Regulating expression and/or function” of a subject gene refers to regulating (increasing or decreasing) an expression level of the subject gene or the function of the subject gene (i.e., inherent function), and is not particularly limited, as long as it does so. “Expression” of a subject gene includes both expression of mRNA of the subject gene and expression of protein of the subject gene, but is preferably expression of protein of the subject gene. “Increase” means that an activity and/or expression level of a subject gene in a sample obtained from a yeast obtained by the production method of a yeast according to the present invention (a yeast at a certain point in time of culture, preferably a yeast at an earlier stage (e.g., elapse of one day) from the start of culture) is higher (e.g., 1.5 times, 2 times, 3 times, 4 times, 5 times, or the like) than the activity and/or expression level of the subject gene in a sample obtained from the original yeast before being subjected to the production method of a yeast according to the present invention. “Decrease” means that an activity and/or expression level of a subject gene in a sample obtained from a yeast obtained by the production method of a yeast according to the present invention (a yeast at a certain point in time of culture, preferably a yeast at an earlier stage (e.g., elapse of one day) from the start of culture) is lower (e.g., ½, ⅕, 1/10, 1/20, 1/50, 1/100, 1/200, 1/500, 1/1000, 1/2000, 1/5000, 1/10000 or less) than the activity and/or expression level of the subject gene in a sample obtained from the original yeast before being subjected to the production method of a yeast according to the present invention. Note that the activity and/or expression level of the subject gene can be measured in accordance with a known method.

The method for regulating expression and/or function of the subject gene is not particularly limited, and a method in accordance with or based on a known method can be adopted. In a case of increasing the expression and/or function of the subject gene, for example, a method such as introducing an expression cassette of the subject gene or modifying an expression control region of the subject gene (for example, substituting a promoter with a promoter having higher activity, introducing a transcription activation element, or the like) can be used. A highly active promoter that can be used as the promoter of the expression cassette or the promoter after substitution is not particularly limited, and examples thereof include a TDH3 promoter, a GAL10 promoter, a CMV promoter, an EF1 promoter, an SV40 promoter, an MSCV promoter, a CAG promoter, and an artificial promoter obtained by artificially combining a transcription activation element and a core promoter. In a case of decreasing the expression and/or function of the subject gene, for example, methods such as deleting the subject gene, introducing mutation into a protein coding region of the subject gene (e.g., introducing a stop codon in the middle of the coding region, introducing mutation causing a frame shift, or the like), introducing mutation into a splicing regulatory region of the subject gene (e.g., introducing mutation into a cis-element of splicing control), introducing mutation into the expression regulatory region of the subject gene (e.g., introducing mutation into a transcription activation element), and the like are exemplified. Among these, gene deletion is preferable. More specifically, the above can be carried out in accordance with or pursuant to a known genetic engineering technique, for example, using a technique such as a CRISPR/Cas system, a TALEN system, siRNA, miRNA, or transformation.

The production method of a yeast according to the present invention preferably includes decreasing the expression and/or function of the yeast 110315 gene. This can produce a yeast with improved oil and/or fat production ability.

The production method of a yeast according to the present invention further preferably includes decreasing the expression of the yeast 110315 gene. This can produce a yeast with improved oil and/or fat production ability.

### 3. Reagent for Production of Yeast with Regulated Oil and/or Fat Production Ability

In one aspect, the present invention relates to a reagent for production of a yeast with regulated oil and/or fat production ability, the reagent containing at least one selected from the group consisting of an expression regulator and a function regulator of a yeast 110315 gene (in the present specification, also referred to as “reagent for production of a yeast according to the present invention”). This will be described below. Note that for matters not described in the present section, the description in “2. Production method of yeast with regulated oil and/or fat production ability” is incorporated herein by reference.

### 3-1. Subject Gene Expression Regulator

The subject gene expression regulator is not particularly limited, as long as it can regulate the expression of the subject gene protein or the subject gene mRNA, and examples thereof include a subject gene expression inhibitor and a subject gene expression promoter. The subject gene expression regulator may be used alone or in a combination of two or more types thereof.

### 3-1-1. Subject Gene Expression Inhibitor

The subject gene expression inhibitor is not particularly limited, as long as it can inhibit the expression level of the subject gene protein, the subject gene mRNA, or the like, and examples thereof include a subject gene-specific small interfering RNA (siRNA), a subject gene-specific microRNA (miRNA), a subject gene-specific antisense nucleic acid, expression vectors thereof; subject gene-specific ribozyme; and a gene editing agent of the subject gene gene by a CRISPR/Cas system.

3-1-1-1. siRNA, miRNA, Antisense Nucleic Acid, and Ribozyme

The subject gene-specific siRNA is not particularly limited, as long as it is a double-stranded RNA molecule that specifically inhibits the expression of the subject gene. In one embodiment, the siRNA preferably has a length of, for example, 18 bases or more, 19 bases or more, 20 bases or more, or 21 bases or more. In addition, the siRNA preferably has a length of, for example, 25 bases or less, 24 bases or less, 23 bases or less, or 22 bases or less. It is contemplated that the upper and lower limits for the length of the siRNA described herein are arbitrarily combined. For example, combinations of lengths having a lower limit of 18 bases and an upper limit of 25 bases, 24 bases, 23 bases, or 22 bases; a lower limit of 19 bases and an upper limit of 25 bases, 24 bases, 23 bases, or 22 bases; a lower limit of 20 bases and an upper limit of 25 bases, 24 bases, 23 bases, or 22 bases; and a lower limit of 21 bases and an upper limit of 25 bases, 24 bases, 23 bases, or 22 bases are contemplated.

The siRNA may be shRNA (small hairpin RNA). The shRNA can be designed in such a manner that a portion thereof forms a stem-loop structure. For example, when the sequence of a certain region is a sequence a and the complementary strand to the sequence a is a sequence b, shRNA can be designed in such a manner that these sequences are present in one RNA strand in the order of the sequence a, a spacer, and the sequence b to have a total length of 45 to 60 bases. The sequence a is a sequence of a partial region of a base sequence encoding a subject gene which is a target, the target region is not particularly limited, and an arbitrary region can be used as a candidate. The length of the sequence a is 19 to 25 bases, and preferably 19 to 21 bases.

The subject gene-specific siRNA may have an additional base at 5′ or 3′ end. The length of the additional base is usually about 2 to 4 bases. The additional base may be DNA or RNA, but when DNA is used, it may be possible to improve the stability of the nucleic acid. Examples of the sequence of such an additional base include, but are not limited to, the following sequences: ug-3′, uu-3′, tg-3′, tt-3′, ggg-3′, guuu-3′, gttt-3′, ttttt-3′, and uuuuu-3′.

The siRNA may have a protruding portion sequence (overhang) at 3′ end, and specific examples thereof include those having dTdT (dT represents deoxythymidine) added thereto. Alternatively, the siRNA may have a flush end (blunt end) without addition at an end. In the siRNA, the sense strand and the antisense strand may have different numbers of bases, and examples of the siRNA include “asymmetrical interfering RNA (aiRNA)” in which the antisense strand has a protruding portion sequence (overhang) at 3′ end and 5′ end. In typical aiRNA, the antisense strand includes 21 bases, the sense strand includes 15 bases, and each of both ends of the antisense strand has an overhang structure of 3 bases.

Although the position of the target sequence of the subject gene-specific siRNA is not particularly limited, in one embodiment, it is desirable to select the target sequence from a region from the 5′-UTR and the initiation codon to about 50 bases, and from a region other than the 3′-UTR. For the candidate group of the selected target sequence, it is preferable to use homology search software such as BLAST (http://www.ncbi.nlm.nih.gov/BLAST/) to examine whether there is homology in a sequence of 16 to 17 bases continuous in mRNA other than the target, thereby confirming the specificity of the selected target sequence. For the target sequence whose specificity has been confirmed, a double-stranded RNA including a sense strand having a 3′-end overhang of TT or UU in 19 to 21 bases after AA (or NA) and an antisense strand having a sequence complementary to the 19 to 21 bases and a 3′-end overhang of TT or UU may be designed as siRNA. Further, shRNA, which is a precursor of siRNA, can be designed by appropriately selecting an arbitrary linker sequence (for example, about 5 to 25 bases) capable of forming a loop structure and linking the sense strand and the antisense strand via the linker sequence.

The sequence of siRNA and/or shRNA can be searched using search software provided free of charge on various websites. Examples of such a site include the following:

siRNA Target Finder (http://www.ambion.com/jp/techlib/misc/siRNA_finder.html) and an insert design tool for pSilencer (trade name) Expression Vector (http://www.ambion.com/jp/techlib/misc/psilencer_converter.html) provided by Ambion; and GeneSeer (http://codex.cshl.edu/scripts/newsearchhairpin.cgi) provided by RNAi Codex.

siRNA can be prepared by synthesizing a sense strand and an antisense strand of a target sequence on mRNA using an automatic DNA/RNA synthesizer, denaturing the strands in an appropriate annealing buffer at about 90 to about 95° C. for about 1 minute, and then annealing the strands at about 30 to about 70° C. for about 1 to about 8 hours. siRNA can also be prepared by synthesizing shRNA serving as a precursor of siRNA and cleaving it with an RNA-cleaving protein dicer.

The subject gene-specific miRNA is arbitrary as long as it inhibits translation of the subject gene. For example, miRNA may pair with a 3′ untranslated region (UTR) of a target to inhibit its translation, rather than cleaving target mRNA as does siRNA. miRNA may be any of pri-miRNA (primary miRNA), pre-miRNA (precursor miRNA), and mature miRNA. The length of miRNA is not particularly limited, and the length of pri-miRNA is usually several hundred to several thousand bases, the length of pre-miRNA is usually 50 to 80 bases, and the length of mature miRNA is usually 18 to 30 bases. In one embodiment, the subject gene-specific miRNA is preferably pre-miRNA or mature miRNA, and more preferably mature miRNA. Such a subject gene-specific miRNA may be synthesized by a known method or purchased from a company that provides synthetic RNA.

The subject gene-specific antisense nucleic acid is a nucleic acid including a base sequence complementary or substantially complementary to the base sequence of mRNA of the subject gene or a part thereof, and having a function of inhibiting the synthesis of the subject gene protein by forming a stable double strand specific to the mRNA and binding thereto. The antisense nucleic acid may be DNA, RNA or a DNA/RNA chimera. In a case where the antisense nucleic acid is DNA, RNA:DNA hybrid formed by the target RNA and the antisense DNA is recognized by endogenous ribonuclease H (RNase H) and causes selective degradation of the target RNA. Thus, in a case of antisense DNA which is directed to degradation by RNase H, the target sequence may be not only a sequence in the mRNA, but also a sequence of an intron region in the initial translation product of the subject gene gene. The intron sequence can be determined by comparing the genomic sequence with the cDNA base sequence of the subject gene gene using a homology search program such as BLAST or FASTA.

The target region of the subject gene-specific antisense nucleic acid is not particularly limited in length as long as it is hybridized with the antisense nucleic acid to thereby inhibit translation into the subject gene protein. The subject gene-specific antisense nucleic acid may be the entire sequence or a partial sequence of mRNA encoding the subject gene. In consideration of ease of synthesis, antigenicity, a problem of intracellular transferability, and the like, an oligonucleotide including about 10 to about 40 bases, particularly about 15 to about 30 bases is preferred, but is not limited thereto. More specifically, a 5′-end hairpin loop, a 5′-end untranslated region, a translation initiation codon, a protein encoding region, an ORF translation termination codon, a 3′-end untranslated region, a 3′-end palindromic region, a 3′-end hairpin loop, or the like of the subject gene gene can be selected as a preferred target region of the antisense nucleic acid, but the target region is not limited thereto.

The subject gene-specific antisense nucleic acid may not only hybridize with mRNA or an initial transcription product of the subject gene gene to inhibit translation into a protein, but also bind to these genes which are double-stranded DNA to form a triple strand (triplex) to inhibit transcription into RNA (antigen).

The subject gene-specific siRNA, the subject gene-specific miRNA, the subject gene-specific antisense nucleic acid, and the like can be prepared by determining a target sequence of mRNA or an initial transcription product based on a cDNA sequence or a genomic DNA sequence of the subject gene gene, and synthesizing a sequence complementary thereto using a commercially available DNA/RNA automatic synthesizer. Antisense nucleic acids containing various types of modification can also be chemically synthesized by a known method.

The expression cassette for the subject gene-specific siRNA, the subject gene-specific miRNA, or the subject gene-specific antisense nucleic acid is not particularly limited, as long as it is a polynucleotide into which the subject gene-specific siRNA, the subject gene-specific miRNA, or the subject gene-specific antisense nucleic acid is incorporated in an expressible state. Typically, the expression cassette includes a polynucleotide including a promoter sequence, and a coding sequence for the subject gene-specific siRNA, the subject gene-specific miRNA, or the subject gene-specific antisense nucleic acid (and a transcription termination signal sequence as necessary), and other sequences as necessary. The promoter is not particularly limited, and examples thereof include RNA polymerase II (polII)-based promoters such as a CMV promoter, an EF1 promoter, an SV40 promoter, an MSCV promoter, an hTERT promoter, a R-actin promoter, and a CAG promoter; and RNA polymerase III (polIII)-based promoters such as mouse and human U6-snRNA promoters, a human H1-RNase P RNA promoter, and a human valine-tRNA promoter. Among these, the polIII-based promoter is preferred from the viewpoint that it is possible to accurately perform transcription of short RNA. Various promoters inducible by a drug can also be used. The other sequences are not particularly limited, and various known sequences that can be contained in an expression vector can be adopted. Examples of such a sequence include a replication origin and a drug resistance gene. The types of the drug resistance gene and the type of the vector can be exemplified by those described above.

Other examples of the subject gene expression inhibitor include subject gene-specific ribozyme. The term “ribozyme” means, in a narrow sense, RNA having an enzymatic activity of cleaving a nucleic acid, but in the present application, also includes DNA as long as it has a sequence-specific nucleic acid cleaving activity. As the most versatile ribozyme nucleic acid, there can be exemplified self-splicing RNA found in infectious RNA such as viroid or virusoid, and a hammerhead type, a hairpin type, and the like are known. The hammerhead type exhibits the enzymatic activity with about 40 bases, and it is possible to specifically cleave only target mRNA by making several bases (about 10 bases in total) at either end adjacent to a portion having the hammerhead structure a sequence complementary to the desired cleavage site of mRNA. This type of ribozyme nucleic acid has an advantage of not attacking genomic DNA because it uses only RNA as a substrate. In a case where mRNA of the subject gene gene has a double-stranded structure by itself, the target sequence can be made single-stranded by using hybrid ribozyme to which an RNA motif derived from viral nucleic acid capable of specifically binding to RNA helicase is linked [Proc. Natl. Acad. Sci. USA, 98(10): 5572-5577 (2001)]. Furthermore, in a case where ribozyme is used in the form of an expression vector containing DNA encoding the ribozyme, the ribozyme can be made hybrid ribozyme by further linking a sequence obtained by modifying tRNA to promote transfer of the transcription product into cytoplasm [Nucleic Acids Res., 29(13): 2780-2788 (2001)].

### 3-1-1-2. Gene Editing Agent

The subject gene gene editing agent is not particularly limited, as long as it can inhibit expression of the subject gene gene by a target sequence-specific nuclease system (e.g., CRISPR/Cas system). The expression of the subject gene gene can be inhibited by, for example, disrupting the subject gene gene or modifying a promoter of the subject gene gene to inhibit activity of the promoter.

As the subject gene gene editing agent, for example, in a case where the CRISPR/Cas system is adopted, typically, a vector (subject gene gene editing vector) including a guide RNA expression cassette targeting the subject gene gene or a promoter thereof and a Cas protein expression cassette can be used, but this is not a limitation. In addition to this typical example, for example, a combination of a guide RNA targeting the subject gene gene or a promoter thereof and/or a vector containing an expression cassette thereof and a Cas protein expression cassette and/or a vector containing an expression cassette thereof can be used as the subject gene gene editing agent.

The guide RNA expression cassette is not particularly limited, as long as it is a polynucleotide used for the purpose of expressing guide RNA in a yeast. Typical examples of the expression cassette include a polynucleotide including a promoter and a coding sequence of the whole or a part of guide RNA placed under control of the promoter. Note that “placed under control of the promoter” means, in other words, that the guide RNA coding sequence is placed in such a manner that transcription of the sequence is controlled by the promoter. As a specific aspect of placement, for example, an aspect in which the guide RNA coding sequence is placed immediately under the 3′ side of the promoter (for example, an aspect in which the number of base pairs (bp) from the base at 3′ end of the promoter to the base at 5′ end of the guide RNA coding sequence is, for example, 100 bp or less, preferably 50 bp or less) can be exemplified.

The promoter of the guide RNA expression cassette is not particularly limited, and a pol II-based promoter can be used, but a pol III-based promoter is preferable from the viewpoint of more accurately performing transcription of relatively short RNA. The pol III-based promoter is not particularly limited, and examples thereof include mouse and human U6-snRNA promoters, a human H1-RNase P RNA promoter, and a human valine-tRNA promoter. Various promoters inducible by a drug can also be used.

The guide RNA coding sequence is not particularly limited, as long as it is a base sequence encoding guide RNA.

The guide RNA is not particularly limited, as long as it is used in the CRISPR/Cas system. For example, it is possible to use various types of guide RNA that bind to a target site of genomic DNA (for example, the subject gene gene, the promoter thereof, or the like) and bind to Cas protein to guide the Cas protein to the target site of the genomic DNA.

In the present specification, the term, target site, refers to a site on genomic DNA which includes a DNA strand (target strand) including a proto-spacer adjacent motif (PAM) sequence and a sequence having a length of about 17 to 30 bases (preferably a length of 18 to 25 bases, more preferably a length of 19 to 22 bases, particularly preferably a length of 20 bases) adjacent to the 5′ side of the PAM sequence and a complementary DNA strand (non-target strand) thereof.

The PAM sequence varies depending on the type of Cas protein utilized. For example, the PAM sequence corresponding to Cas9 protein (type II) derived from S. pyogenes is 5′-NGG, the PAM sequence corresponding to Cas9 protein (type I-A1) derived from S. solfataricus is 5′-CCN, the PAM sequence corresponding to Cas9 protein (type I-A2) derived from S. solfataricus is 5′-TCN, the PAM sequence corresponding to Cas9 protein (type I-B) from H. walsbyl is 5′-TTC, the PAM sequence corresponding to Cas9 protein (type I-E) derived from E. coli is 5′-AWG, the PAM sequence corresponding to Cas9 protein (type I-F) derived from E. coli is 5′-CC, the PAM sequence corresponding to Cas9 protein (type I-F) derived from P. aeruginosa is 5′-CC, the PAM sequence corresponding to Cas9 protein (type II-A) derived from S. thermophilus is 5′-NNAGAA, the PAM sequence corresponding to Cas9 protein (type II-A) derived from S. agalactiae is 5′-NGG, the PAM sequence corresponding to Cas9 protein derived from S. aureus is 5′-NGRRT or 5′-NGRRN, the PAM sequence corresponding to Cas9 protein derived from N. meningitidis is 5′-NNNNGATT, and the PAM sequence corresponding to Cas9 protein derived from T. denticola is 5′-NAAAAC.

The guide RNA has a sequence (also referred to as a crRNA (CRISPR RNA) sequence) involved in binding to the target site of the genomic DNA, and the crRNA sequence binds complementarily (preferably complementarily and specifically) to a sequence excluding the PAM sequence-complementary sequence of the non-target strand, whereby the guide RNA can bind to the target site of the genomic DNA.

Note that “complementarily” binding includes not only binding based on complete complementarity (A and T, and G and C), but also binding based on complementarity to the extent that hybridization can be achieved under stringent conditions. The stringent conditions can be determined based on the melting temperature (Tm) of a complex or nucleic acid to which a probe binds, as taught in Berger and Kimmel (1987, Guide to Molecular Cloning Techniques Methods in Enzymology, Vol. 152, Academic Press, San Diego CA). For example, as washing conditions after hybridization, conditions of about “1×SSC, 0.1% SDS, 37° C.” can be usually exemplified. It is preferable that the hybridized state is maintained even when washing is performed under such conditions. Although not particularly limited, examples of more stringent hybridization conditions include washing conditions of about “0.5×SSC, 0.1% SDS, 42° C.”, and examples of even more stringent hybridization conditions include washing conditions of about “0.1×SSC, 0.1% SDS, 65° C.”.

Specifically, among the crRNA sequences, the sequence that binds to the target sequence has, for example, 90% or more, preferably 95% or more, more preferably 98% or more, even more preferably 99% or more, particularly preferably 100% identity with the target strand. Note that for the binding of the guide RNA to the target site, 12 bases on the 3′ side of the sequence that binds to the target sequence in the crRNA sequence are said to be important. Accordingly, in a case where among the crRNA sequences, the sequence that binds to the target sequence is not completely identical to the target strand, a base that is different from the target strand is preferably present at a position other than 12 bases on the 3′ side of the sequence that binds to the target sequence in the crRNA sequence.

The guide RNA has a sequence involved in binding to the Cas protein (also referred to as a tracrRNA (trans-activating crRNA) sequence), and the tracrRNA sequence can guide Cas protein to the target site of the genomic DNA by binding to the Cas protein.

The tracrRNA sequence is not particularly limited. The tracrRNA sequence is typically RNA including a sequence having a length of about 50 to 100 bases that can form a plurality of (usually three) stem loops, and the sequence varies depending on the type of Cas protein to be used.

As the tracrRNA sequence, various known sequences can be adopted depending on the type of Cas protein to be used.

The guide RNA typically includes the crRNA sequence and the tracr RNA sequence as described above. An aspect of the guide RNA may be single-stranded RNA (sgRNA) including the crRNA sequence and the tracr RNA sequence, or may be an RNA complex in which RNA including the crRNA sequence and RNA including the tracrRNA sequence are complementarily bound to each other.

The Cas protein expression cassette is not particularly limited, as long as it is a polynucleotide used for the purpose of expressing Cas protein in an organism whose metabolism is to be improved. Typical examples of the expression cassette include a polynucleotide including a promoter and a Cas protein coding sequence placed under control of the promoter. Note that “placed under control of the promoter” is the same as the definition in the guide RNA expression cassette.

The promoter of the Cas protein expression cassette is not particularly limited, and for example, various pol II-based promoters can be used. The pol II-based promoter is not particularly limited, and examples thereof include a TDH3 promoter, a GAL10 promoter, a CMV promoter, an EF1 promoter, an SV40 promoter, an MSCV promoter, and a CAG promoter. Various promoters inducible by a drug can also be used.

The Cas protein coding sequence is not particularly limited, as long as it is a base sequence encoding the amino acid sequence of Cas protein.

The Cas protein is not particularly limited, as long as it is used in the CRISPR/Cas system.

For example, various types of Cas protein capable of binding to a target site of genomic DNA in a state of forming a complex with the guide RNA and cleaving the target site can be used. Cas protein derived from various organisms is known, and examples thereof include Cas9 protein (type II) derived from S. pyogenes, Cas9 protein (type I-A1) derived from S. solfataricus, Cas9 protein (type I-A2) derived from S. solfataricus, Cas9 protein (type I-B) derived from H. walsbyl, Cas9 protein (type I-E) derived from E. coli, Cas9 protein (type I-F) derived from E. coli, Cas9 protein (type I-F) derived from P. aeruginosa, Cas9 protein (type II-A) derived from S. thermophilus, Cas9 protein (type II-A) derived from S. agalactiae, Cas9 protein derived from S. aureus, Cas9 protein derived from N. meningitidis, Cas9 protein derived from T. denticola, and Cpf1 protein (type V) derived from F. novicida. Among them, Cas9 protein is preferred, and Cas9 protein which bacteria belonging to the genus Streptococcus endogenously have is more preferred. Information on amino acid sequences of various types of Cas protein and coding sequences thereof can be easily obtained on various databases such as NCBI.

The Cas protein may be wild-type double-stranded truncated Cas protein or nickase-type Cas protein. In addition, the Cas protein may have mutation (for example, substitution, deletion, insertion, addition, or the like) of the amino acid sequence, or may be one to which a known protein tag, a signal sequence, or protein such as enzyme protein is added, as long as the activity thereof is not impaired. Examples of the protein tag include biotin, His tag, FLAG tag, Halo tag, MBP tag, HA tag, Myc tag, V5 tag, and PA tag. Examples of the signal sequence include a cytoplasmic localization signal.

The subject gene gene editing vector may have other sequences. The other sequences are not particularly limited, and various known sequences that can be contained in an expression vector can be adopted. Examples of such a sequence include a replication origin and a drug resistance gene.

Examples of the drug resistance gene include a chloramphenicol resistance gene, a tetracycline resistance gene, a neomycin resistance gene, an erythromycin resistance gene, a spectinomycin resistance gene, a kanamycin resistance gene, a hygromycin resistance gene, and a puromycin resistance gene.

The type of the vector is not particularly limited, and examples thereof include plasmid vectors such as animal cell expression plasmid; viral vectors such as retrovirus, lentivirus, adenoviruses, adeno-associated virus, herpes virus, and sendai virus; and Agrobacterium vector.

The subject gene gene editing agent can be easily prepared in accordance with a known genetic engineering technique. For example, PCR, restriction enzyme fragmentation, DNA ligation technology, in vitro transcription/translation technology, recombinant protein production technology, or the like can be used to produce the subject gene editing agent.

### 3-1-2. Subject Gene Expression Promoter

The subject gene expression promoter is not particularly limited, as long as it can increase the amount of the subject gene in a cell.

Examples of the subject gene expression promoter include an expression cassette of the subject gene. The expression cassette of the subject gene is not particularly limited, as long as the subject gene is incorporated in an expressible state. Typically, the expression cassette of the subject gene includes a polynucleotide including a promoter sequence and a coding sequence for the subject gene (and optionally a transcription termination signal sequence). The expression cassette can also be in the form of a vector.

The expression vector is not particularly limited, and examples thereof include plasmid vectors such as an animal cell expression plasmid; and viral vectors such as retrovirus, lentivirus, adenovirus, adeno-associated virus, herpesvirus, and sendai virus.

The promoter is not particularly limited, and examples thereof include a TDH3 promoter, a GAL10 promoter, a CMV promoter, an EF1 promoter, an SV40 promoter, an MSCV promoter, and a CAG promoter. Various promoters inducible by a drug can also be used.

In addition to the above, the expression vector may contain other elements that can be contained in the expression vector. Examples of the other elements include a replication origin and a drug resistance gene. The drug resistance gene is not particularly limited, and examples thereof include a chloramphenicol resistance gene, a tetracycline resistance gene, a neomycin resistance gene, an erythromycin resistance gene, a spectinomycin resistance gene, a kanamycin resistance gene, a hygromycin resistance gene, and a puromycin resistance gene.

The expression vector of the subject gene can be easily obtained in accordance with a known genetic engineering technique. For example, it can be prepared using PCR, restriction enzyme fragmentation, DNA ligation technology, or the like.

Other examples of the subject gene expression promoter include a transcription activator of the subject gene and an expression vector thereof, and a low molecular compound capable of activating transcription of the subject gene. The aspect of the expression vector is the same as that of the expression vector of the subject gene.

### 3-2. Subject Gene Function Regulator

The subject gene function regulator is not particularly limited, as long as it can regulate the function of the subject gene protein or the subject gene mRNA, and examples thereof include a subject gene function inhibitor and a subject gene function promoter. The subject gene function regulator may be used alone or in a combination of two or more types thereof.

Examples of the subject gene function regulator include a neutralizing antibody against the subject gene protein. The neutralizing antibody refers to an antibody having a property of inhibiting the activity of the subject gene protein by binding to the subject gene.

Examples of the antibody include a polyclonal antibody, a monoclonal antibody, a chimeric antibody, a single-chain antibody, and a part of the antibody having an antigen-binding property, such as Fab fragments or fragments produced by a Fab expression library. The antibody of the present invention also includes an antibody having an antigen-binding property to polypeptide including at least, usually 8 amino acids, preferably 15 amino acids, and more preferably 20 amino acids, which are continuous, in the amino acid sequence of the subject gene.

As the neutralizing antibody, for example, an antibody having an antigen-binding property to an amino acid sequence of a binding site with another molecule (for example, nucleic acid, protein, substrate, or the like) in the subject gene protein is preferable. The binding site can be determined based on known information and/or inferred based on known information (e.g., by docking model construction, etc.).

Methods for production of these antibodies are already well known, and the antibody of the present invention can also be produced in accordance with these common methods (Current protocols in Molecular Biology, Chapters 11.12 to 11.13 (2000)). Specifically, in a case where the antibody of the present invention is a polyclonal antibody, it can be obtained as follows: a subject gene, which is expressed in E. coli or the like and purified in accordance with a common method, or an oligopeptide having a partial amino acid sequence of the subject gene, which is synthesized in accordance with a common method, is used to immunize a non-human animal such as a rabbit, and the antibody is obtained from the serum of the immunized animal in accordance with a common method. On the other hand, in a case where the antibody is a monoclonal antibody, it can be obtained from hybridoma cells prepared as follows: a non-human animal such as a mouse is immunized with a subject gene expressed in E. coli or the like and purified in accordance with a common method, or an oligopeptide having a partial amino acid sequence of the subject gene, and the resulting spleen cells are fused with the resulting myeloma cells to prepare the hybridoma cells (Current protocols in Molecular Biology edit. Ausubel et al. (1987) Publish. John Wiley and Sons. Sections 11.4 to 11.11).

The subject gene to be used as an immunogen in preparing the antibody can be obtained by DNA cloning, construction of each plasmid, transfection into a host, culture of a transformant, and recovery of protein from the culture on the basis of known gene sequence information. These operations can be carried out pursuant to a method known to those skilled in the art, a method described in literature (Molecular Cloning, T. Maniatis et al., CSH Laboratory (1983), DNA Cloning, DM. Glover, IRL PRESS (1985)), or the like.

Specifically, protein as an immunogen for producing the antibody of the present invention can be obtained by preparing recombinant DNA (expression vector) that can express the subject gene in a desired host cell, introducing the recombinant DNA into the host cell to transform the host cell, culturing the transformant, and recovering the target protein from the resulting culture. Partial peptide of the subject gene can also be produced by a general chemical synthesis method (peptide synthesis) in accordance with known gene sequence information.

The antibody of the present invention may be prepared using an oligopeptide having a partial amino acid sequence of the subject gene. The oligo(poly)peptide to be used for producing such an antibody does not need to have functional biological activity, but preferably has an immunogenic property similar to that of the subject gene. An oligo(poly)peptide preferably having this immunogenic property and including at least 8 contiguous amino acids, preferably 15 amino acids, more preferably 20 amino acids in the amino acid sequence of the subject gene can be exemplified.

An antibody against such an oligo(poly)peptide can also be produced by enhancing immunological response using various adjuvants depending on the host. Examples of such adjuvants include, but are not limited to, a Freund's adjuvant; mineral gel such as aluminum hydroxide; surface active substances such as lysolecithin, pluronic polyol, polyanion, peptide, oil emulsion, keyhole limpet hemocyanin, and dinitrophenol; and human adjuvants such as BCG (bacille Calmette-Guerin) and Corynebacterium parvum.

As the subject gene function regulator, in addition to the above-mentioned subject gene neutralizing antibody, a subject gene antagonist, a subject gene agonist, a subject gene dominant negative mutant, or the like can be used. In a case where protein such as a neutralizing antibody is employed as the subject gene function regulator, an expression cassette thereof can be employed instead. The expression cassette is as defined in “3-1. Subject gene expression regulator” above.

### 3-3. Others

The reagent for production of a yeast according to the present invention is used for production of a yeast with regulated oil and/or fat production ability. Specifically, the reagent for production of a yeast according to the present invention is introduced into a yeast to regulate expression or the like of the subject gene in the yeast or to express an exogenous gene, thereby producing a yeast with regulated oil and/or fat production ability.

The reagent for production of a yeast according to the present invention is preferably a reagent for use in producing a yeast with improved oil and/or fat production ability, which contains at least one selected from the group consisting of an expression inhibitor and a function inhibitor of the yeast 110315 gene.

The reagent for production of a yeast according to the present invention is more preferably a reagent for use in producing a yeast with improved oil and/or fat production ability, which contains an expression inhibitor of the yeast 110315 gene.

The reagent for production of a yeast according to the present invention may consist only of the above-described essential components, but may contain various other components in addition to the essential components depending on types of the essential components to be contained, a dosage form, a mode of use, and the like described below. The content ratio of the essential components (dry weight) in an agent for production of the present invention can be appropriately determined depending on the dosage form, the mode of use, and the like described below, and can be, for example, in a range of 0.0001 to 100 mass %. Examples of the other components include a base, a carrier, a solvent, a dispersant, an emulsifier, a buffer, a stabilizer, an excipient, a binder, a disintegrant, a lubricant, a thickener, a humectant, a colorant, a perfume, and a chelating agent. The form of the agent for production according to the present invention is not particularly limited, and may be, for example, a dry form, a solution form, or the like, and may be a kit form. As necessary, the kit may appropriately include other materials necessary for yeast culture, such as a nucleic acid introduction reagent or a buffer solution, reagents, instruments, and the like.

### 4. Oil and/or Fat Producing Yeast, Oil and/or Fat Producing Composition, and Production Method of Oil and/or Fat

In one aspect, the present invention relates to an oil and/or fat producing yeast in a state in which expression of the yeast 110315 gene is reduced (in the present specification, also referred to as the “oil and/or fat producing yeast according to the present invention”).

In addition, in one aspect, the present invention relates to a composition for producing an oil and/or fat (in the present specification, also referred to as the “composition for producing an oil and/or fat according to the present invention”) containing the oil and/or fat producing yeast according to the present invention.

Furthermore, in one aspect, the present invention relates to a production method of an oil and/or fat (in the present specification, also referred to as the “production method of an oil and/or fat according to the present invention”) including recovering an oil and/or fat from at least one selected from the group consisting of a culture of the oil and/or fat producing yeast according to the present invention and the composition for producing an oil and/or fat according to the present invention.

These will be described below. Note that for matters not described in the present section, the description in “2. Production method of yeast with regulated oil and/or fat production ability” is incorporated herein by reference.

The oil and/or fat producing yeast according to the present invention is a yeast that is obtained by the production method of a yeast according to the present invention, and is not particularly limited, as long as it is obtained.

The oil and/or fat producing yeast according to the present invention may be further subjected to mutation. For example, the oil and/or fat producing yeast according to the present invention may be subjected to mutation in a fatty acid conversion pathway. To be more specific, for example, C16/C18 fatty-acid elongase, Δ9 desaturase, Δ9 elongase, Δ8 desaturase, Δ5 desaturase, Δ15 desaturase, Δ17 desaturase, or the like may be subjected to mutation in the fatty acid conversion pathway by mutation such as introduction of an exogenous gene or modification of an endogenous gene or a promoter thereof. This makes it possible to further increase, for example, a polyunsaturated fatty acid content, preferably a content of polyunsaturated fatty acid with high added value, such as DHA or EPA.

The composition for producing an oil and/or fat according to the present invention is not particularly limited, as long as it contains the oil and/or fat producing yeast according to the present invention. The composition for producing an oil and/or fat according to the present invention can be, for example, a culture of the oil and/or fat producing yeast according to the present invention or a suspension of the oil and/or fat producing yeast according to the present invention.

The culture can be performed by a method known in the related art using a culture solution containing a carbon source. As the carbon source, saccharides, sugar alcohols, acidic sugars, or biomass containing these can be used without particular limitation. Herein, “biomass” in the present invention is understood to mean a renewable material containing the carbon source.

Examples of the saccharides include monosaccharides, oligosaccharides, and polysaccharides. The term “oligosaccharides” refers to a disaccharide to a decasaccharide, and these may be a homooligosaccharide or a heterooligosaccharide. The term “polysaccharides” refers to saccharides having a larger number of monosaccharide units than that of oligosaccharides, and these may be a homopolysaccharide or a heteropolysaccharide. Specific examples of the monosaccharides include pentoses such as L-arabinose, D-xylose, and D-ribose; hexoses such as D-glucose, D-galactose, D-fructose, and D-mannose; and 6-deoxyhexoses such as L-rhamnose. Examples of the oligosaccharides include disaccharides such as sucrose, maltose, lactose, cellobiose, trehalose, and melibiose; and trisaccharides such as raffinose. Examples of the polysaccharides include starch, cellulose, glycogen, dextran, mannan, and xylan. The above saccharides may be used alone or in an appropriate combination. Starch hydrolysate and the like are also included in the above combination. As the saccharides, a raw material containing a saccharide as a main component, such as molasses or bean curd refuse, can also be used.

Examples of the sugar alcohols include D-sorbitol, D-mannitol, galactitol, and maltitol. Examples of the acidic sugars include glucuronic acid and galacturonic acid.

The amount of the carbon source in the medium is not particularly limited, and is usually about 3 to 15% (w/w).

The medium may contain, in addition to the carbon source, a nitrogen source, an inorganic substance, and other nutrients. As the nitrogen source, an inorganic organic nitrogen compound such as ammonia, ammonium chloride, ammonium sulfate, ammonium carbonate, ammonium acetate, sodium nitrate, or urea can be used. Further, as the nitrogen source, a nitrogen-containing natural substance such as peptone, meat extract, yeast extract, corn steep liquor, casein hydrolysate, fish meal or a digest thereof, or defatted soybean meal or a digest thereof can also be used. Examples of the inorganic substance include monopotassium phosphate, dipotassium phosphate, magnesium sulfate, sodium chloride, ferrous sulfate, ferrous chloride, manganese sulfate, calcium chloride, calcium carbonate, zinc sulfate, copper sulfate, ammonium borate/molybdate, and potassium iodide.

One aspect of the culture conditions is as follows. The culture is carried out under aerobic conditions such as shaking culture or submerged stirring culture. The culture temperature is generally preferably 20 to 35° C. However, other temperature conditions may be used as long as the temperature is a temperature at which bacteria can grow. The pH of the medium during the culture is usually 4.0 to 7.2. The culture period is not particularly limited and is, for example, 2 to 10 days.

The obtained culture and cells in the culture contain an oil and/or fat such as oleic acid, palmitic acid, stearic acid, linoleic acid, or palmitoleic acid.

The oil and/or fat can be recovered from the culture in accordance with or pursuant to a known method. For example, it can be recovered by pressing, French press, ball mill, or the like.

The oil and/or fat accumulated in the cells can be recovered, for example, by removing a liquid fraction from the culture as necessary and obtaining an oil and/or fat-containing extract from the resulting cells in accordance with or pursuant to a known method. The liquid fraction can be removed by an operation such as centrifugation or static sedimentation, or an apparatus such as a separator, a decanter, or filtration.

An oil and/or fat secreted outside the cells can be recovered by adding a solvent to the culture or, as necessary, to a liquid fraction obtained by removing the cells from the culture to dissolve the oil and/or fat in the solvent. As the solvent, an organic solvent which dissolves the oil and/or fat, has no or poor miscibility with water, and is liquid at room temperature, such as halogenated lower alkane (chloroform, methylene chloride, carbon tetrachloride, 1,2-dichloroethane), n-hexane, ethyl ether, ethyl acetate, or aromatic hydrocarbon (benzene, toluene, xylene), is suitably used. The amount of the extraction solvent to be added is not particularly limited, as long as the oil and/or fat produced and accumulated in the culture or the liquid fraction thereof can be sufficiently recovered.

### EXAMPLES

The present invention will be described in detail below by way of examples, but is not limited to these examples.

### Test Example 1. Acquisition of Oil and/or Fat Accumulated Mutant Strain

To obtain an oil and/or fat accumulated mutant strain, ethylmethane sulfonic acid as a mutagen or UV was allowed to act on L. starkeyi CBS1807 strain to induce mutation, and the resulting mutant strain group was cultured and fractionated by a Percoll density gradient centrifugation method. The density of oil is lower than that of water, and thus it is expected that the density of high oil and/or fat accumulated cells is different from that of low oil and/or fat accumulated cells, and the high oil and/or fat accumulated cells are fractionated into a low-density fraction and the low oil and/or fat accumulated cells are fractionated into a high-density fraction. It is possible to concentrate high oil and/or fat accumulated mutant cells by repeating the operation of separating the low-density fraction, then culturing the fraction, and re-fractionating the fraction by the density gradient centrifugation method (Patent Document 1).

Thereafter, the solution of each concentrated fraction was spread on a medium plate and the cells were isolated as colonies. The obtained colonies were subjected to liquid culture, and oil and/or fat accumulated mutant strains were screened by combining three evaluations: (1) evaluation of oil and/or fat productivity by flow cytometry after fluorescent staining of oil and/or fat, (2) evaluation of fat globule size by microscopic observation, and (3) quantification of oil and/or fat. As a result, high oil and/or fat accumulated mutant strains E15, E47, A42, K13, and K14 were obtained which have high oil and/or fat productivity as compared with the wild-type strain (Non-Patent Document 1). In the comparative culture of the wild-type strain and the high oil and/or fat accumulated mutant strains, the fat globule sizes in the cells of the wild-type strain and in the high oil and/or fat accumulated mutant strains E15, E47, A42, K13, and K14 did not change so much on the first day of culture, but on the third day of culture, the fat globules in the cells of all the high oil and/or fat accumulated mutant strains were larger than those of the wild-type strain, and the fat globule sizes in E15 and K14 were particularly prominent.

On the third day of culture, the TAG production amounts per cell of the high oil and/or fat accumulated mutant strains E15, E47, A42, K13, and K14 were 2.0, 1.4, 1.5, 1.9, and 2.3 times that of the wild-type strain, respectively, and the TAG production amounts of E15 and K14 were two times or more that of the wild-type strain (Non-Patent Document 1).

Further, in the present study, mutation was induced in the E15 strain having the highest oil and/or fat production ability, thereby attempting to obtain a mutant strain having further improved oil and/or fat productivity, and through the same condensation and screening as described above, mutant strains E15-11, E15-15, and E15-25 were acquired in which the amount of accumulated oil and/or fat per medium was improved to about 3.5 times that of the wild-type strain and about 2 times that of the E15 strain.

Furthermore, a strain (m115694) in which the wild-type 115694 gene of the Alslig4 strain was substituted with missense mutation (g.3413 A>G) derived from E15-11, E15-15, and E15-25 of the oil and/or fat production control factor Transcript Id:115694 (115694) gene found in the related art (Patent Document 2) was acquired by an electroporation method. To acquire a new high oil and/or fat accumulated mutant strain, a mutant strain group obtained by allowing UV to act on the m115694 strain to induce mutation was cultured, and fractionated by the Percoll density gradient centrifugation method. The density of oil is lower than that of water, and thus it is expected that the density of high oil and/or fat accumulated cells is different from that of low oil and/or fat accumulated cells, and the high oil and/or fat accumulated cells are fractionated into a low-density fraction and the low oil and/or fat accumulated cells are fractionated into a high-density fraction. By repeating the operation of separating the low-density fraction, then culturing the fraction, and re-fractionating the fraction by the density gradient centrifugation method, it is possible to concentrate the high oil and/or fat accumulated mutant cells. Thereafter, the solution of the concentrated fraction was spread on a medium plate and the cells were isolated as colonies. The obtained colonies were subjected to liquid culture, and oil and/or fat accumulated mutant strains were screened by oil and/or fat quantification. As a result, a novel high oil and/or fat accumulated mutant strain m115694-19 was acquired, which has high oil and/or fat productivity as compared with m115694.

### Test Example 2. Acquisition of Factor Controlling Productivity of Oil and/or Fat Using Comparative Genomic Analysis

To acquire a factor that improves oil and/or fat productivity, a mutant gene was extracted by comparing genomic base sequences of the strain (m115694) in which the wild-type 115694 gene of the Δlslig4 strain was substituted and the oil and/or fat accumulated mutant strain m115694-19. In performing the comparative genomic analysis, attention was paid to nonsense mutation, frameshift mutation, splicing abnormality, and missense mutation. As a result, Transcript Id: 110315 (https://mycocosm.jgi.doe.gov/Lipst1_1/Lipst1_1.home.html) was found as a mutant gene. The mutant Transcript Id: 110315 found in the oil and/or fat accumulated mutant strain m115694-19 indicated nonsense mutation. Specifically, the nonsense mutation is mutation in which the 1270th base of the DNA sequence of the 110315 gene (SEQ ID NO: 1: including intron, from the initiation codon to the termination codon) was substituted from T to A.

### Test Example 3. Analysis of 110315 Gene-Deleted Strain

Using the wild-type strain (WT) and the Δlslig4 strain as control strains, to clarify the association with TAG production in the 110315 deletion (Δlslig4Δ110315) strain, these strains were prepared. The strains were prepared with reference to the method described in Patent Document 2. Each of the resulting strain and the control strains was added to a 200 mL baffled flask and cultured in an S medium on a 75 mL scale (0.5% (NH₄)₂SO₄, 0.1% KH₂PO₄, 0.01% NaCl, 0.1% yeast extract, 0.05% MgSO₄·7H₂O, 0.01% CaCl₂·2H₂O, 5% glucose) under conditions of 30° C. and 160 rpm for 5 days, and the phenotype was analyzed.

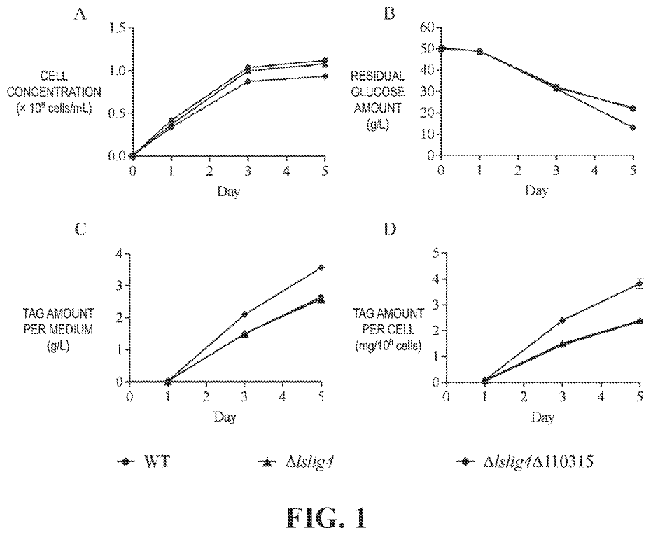

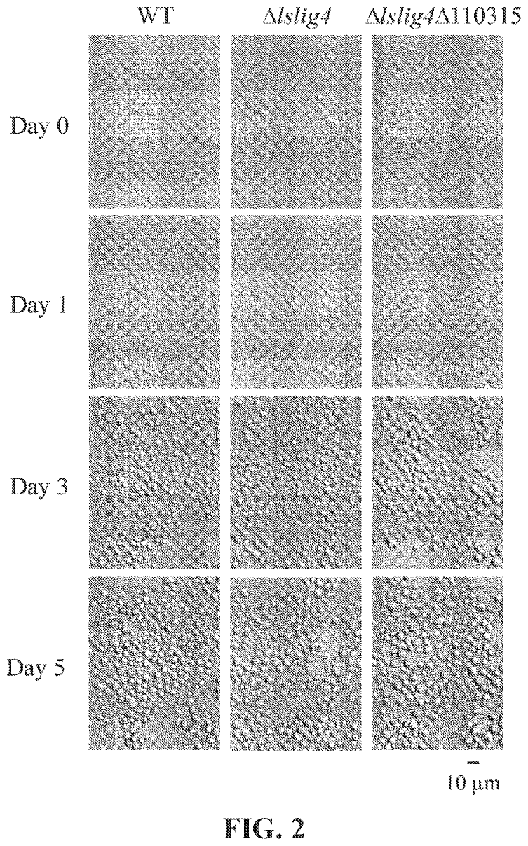

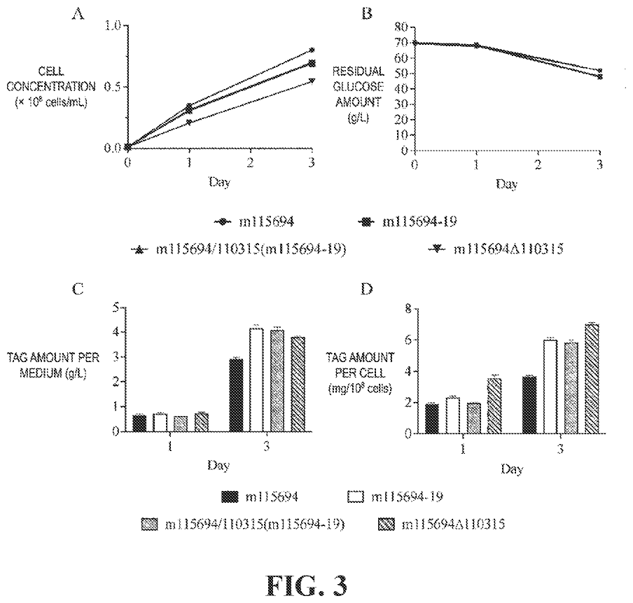

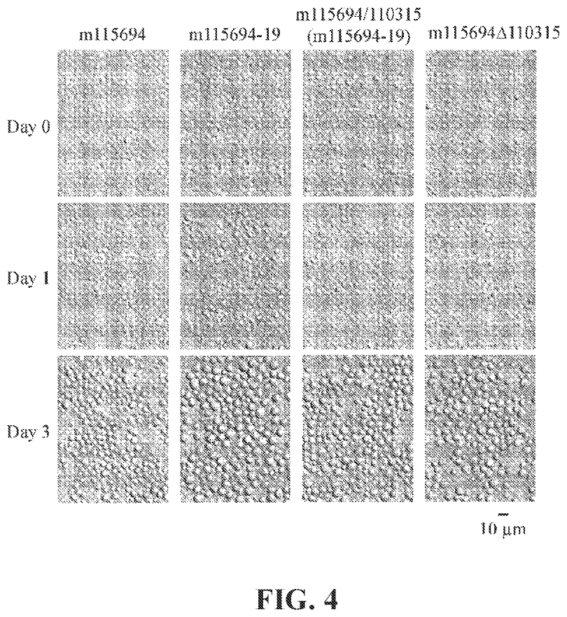

The results are presented in FIGS. 1-1 and 1-2 and Table 1. At the cell concentration, Δlslig4Δ110315 was about 0.9 times the final cell concentration of Δlslig4. In addition, all strains indicated the same level of glucose consumption in the medium until the third day of culture, but on the fifth day of culture, Δlslig4Δ110315 consumed more glucose than WT and Δlslig4 did. Δlslig4Δ110315 produced from 1.3 to 1.5 times as much TAG as the Δlslig4 strain that is the parent strain on and after the third day of culture. Microscopic observation also confirmed that Δlslig4Δ110315 had larger fat globules in the cells than those of Δlslig4 from the third day of culture, indicating high production of TAG. Analysis of the fatty acid composition of the accumulated TAG indicated that Δlslig4Δ110315 had an approximately from 3 to 4% increase in palmitic acid content and an approximately from 2 to 3% decrease in oleic acid content as compared with Δlslig4, but the major fatty acid species in both the strains included palmitic acid and oleic acid.

From the above, it was found that regulating the expression and/or function of the yeast 110315 gene in the yeast can regulate the oil and/or fat production ability.

TABLE 1
Fatty acid compositions of WT, Δlslig4, and Δlslig4Δ110315 at the fifth day of culture

| | Palmitic | Palmitoleic | Stearic | Oleic | Linoleic | Other |
|---|---|---|---|---|---|---|
| Strain | acid (C16:0) | acid (C16:1) | acid (C18:0) | acid (C18:1) | acid (C18:2) | fatty acids |
| WT | 38.15 ± 0.12 | 2.43 ± 0.04 | 8.25 ± 0.19 | 44.10 ± 0.28 | 2.55 ± 0.06 | 4.52 ± 0.06 |
| Δlslig4 | 37.65 ± 0.12 | 2.37 ± 0.06 | 8.61 ± 0.26 | 44.17 ± 0.19 | 2.55 ± 0.15 | 4.65 ± 0.06 |
| Δlslig4Δ110315 | 41.27 ± 0.16 | 2.47 ± 0.03 | 7.90 ± 0.15 | 41.21 ± 0.23 | 3.22 ± 0.05 | 3.93 ± 0.01 |

TAG extracted from cell lysate on the fifth day of culture was subjected to GC-FID. Other fatty acids include C14:0, C15:0, C17:0, C17:1, C18:3n6, C18:3n3, C20:0, C20:1, C22:0, C23:0, C24:0, C24:1n9, and C25:0. Results represent a mean and the standard error of three independent results.

### Test Example 4: Analysis of Strain in which Mutation of 115694 Gene and Mutation and/or Deletion of 110315 Gene are Combined

Using the m115697 strain as a control strain, to clarify the association with TAG production in introduction of mutation derived from the high oil and/or fat accumulated mutant strain m115697-19 into the 110315 gene of the m115697 strain (m115697/110315 (m115697-19)) or deletion of the 110315 gene of the m115697 strain (m115697Δ110315), these strains were prepared. The strains were prepared with reference to the method described in Patent Document 2. Each of the resulting strains and the control strain was added to a 200 mL baffled flask and cultured in an S medium on a 75 mL scale (0.5% (NH₄)₂SO₄, 0.1% KH₂PO₄, 0.01% NaCl, 0.1% yeast-extract, 0.05% MgSO₄·7H₂O, 0.01% CaCl₂·2H₂O, 7% glucose) under conditions of 30° C. and 160 rpm for 3 days, and the phenotype was analyzed.

The results are presented in FIGS. 2-1 and 2-2 and Table 2. On both the first and third days of culture, mutation or deletion of the 110315 gene resulted in a low cell concentration as compared with the control. In addition, m115694-19 and m1115694/110315 (m115697-19) indicated similar cell growth behavior. The glucose consumption amount in the medium was similar from the zeroth day to the first day of culture, but m115694-19 and ml 115694/110315 (m115697-19) consumed slightly more glucose from the first day to the third day of culture. An increase in amount of TAG in the medium was confirmed for all the strains from the first day to the third day of culture, but mutation or deletion of 115694 resulted in a particularly large increase in amount of TAG, and m115694-19 and m1115694/110315 (m115697-19) indicated a TAG production amount of about 1.4 times that of m115694, and m115697Δ110315 indicated a TAG production amount of about 1.3 times. The amount of TAG per cell in m115697Δ110315, which had the lowest cell concentration, was the highest on both the first and third days of culture, and was about 1.9 and 1.8 times that in m115694, respectively. The cell concentration was lower than that of m115694 and the glucose consumption amount was about the same, and thus it is considered that glucose is preferentially used for TAG production rather than growth. In addition, m115694-19 and m1115694/110315 (m115697-19) indicated similar amounts of TAG production per cell. In microscopic observation, it was confirmed that m115694-19, m1115694/110315 (m115697-19), and m115697Δ110315 had many cells having fat globules larger than those of m115697. Analysis of the fatty acid composition of the accumulated TAG indicated that the major fatty acid species in all the strains include palmitic acid and oleic acid, and no significant change was observed in the above fatty acid species.

From the above, it was revealed that the combination of the 115694 gene and the 110315 mutation or 110315 deletion derived from the high oil and/or fat accumulated mutant strain m115694-19 using the related art further improves the TAG production amount.

TABLE 2
Fatty acid compositions of m115694, m115694-19, m115694/110315
(m115694-19), and m115694Δ110315 on the third of culture

| | Palmitic | Palmitoleic | Stearic | Oleic | Linoleic | Other |
|---|---|---|---|---|---|---|
| Strain | acid (C16:0) | acid (C16:1) | acid (C18:0) | acid (C18:1) | acid (C18:2) | fatty acids |
| M115694 | 40.51 ± 0.50 | 2.91 ± 0.08 | 6.11 ± 0.32 | 44.91 ± 0.39 | 2.47 ± 0.17 | 3.09 ± 0.04 |
| M115694-19 | 39.53 ± 0.36 | 2.37 ± 0.05 | 6.95 ± 0.13 | 45.80 ± 0.21 | 2.47 ± 0.08 | 2.87 ± 0.02 |
| M115694/110315 | 40.90 ± 0.80 | 2.94 ± 0.18 | 5.60 ± 0.35 | 44.61 ± 0.69 | 3.07 ± 0.27 | 2.87 ± 0.20 |
| (M115694-19) | | | | | | |
| M115694Δ110315 | 40.84 ± 0.17 | 2.94 ± 0.19 | 5.73 ± 0.42 | 44.82 ± 0.38 | 2.64 ± 0.14 | 3.03 ± 0.12 |

TAG extracted from cell lysate on the third day of culture was subjected to GC-FID. Other fatty acids include C14:0, C15:0, C17:0, C17:1, C18:3n6, C18:3n3, C20:0, C20:1, C22:0, C23:0, C24:0, C24:1n9, and C25:0. Results represent a mean and the standard error of three independent results.

1. A production method of a yeast with regulated oil and/or fat production ability, the production method comprising regulating expression and/or function of a yeast 110315 gene in a yeast.

2. The production method according to claim 1, comprising reducing expression and/or function of the yeast 110315 gene, and producing a yeast with improved oil and/or fat production ability.

3. The production method according to claim 1, comprising reducing expression of the yeast 110315 gene, and producing a yeast with improved oil and/or fat production ability.

4. The production method according to claim 1, wherein the yeast is an oleaginous yeast.

5. The production method according to claim 1, wherein the yeast is a yeast belonging to the genus Lipomyces.

6. A reagent for production of a yeast with regulated oil and/or fat production ability, the reagent comprising at least one selected from the group consisting of an expression regulator and a function regulator of a yeast 110315 gene.

7. The reagent for production according to claim 6, wherein the reagent comprises at least one selected from the group consisting of an expression inhibitor and a function inhibitor of the yeast 110315 gene and is for use in the production of a yeast with improved oil and/or fat production ability.

8. The reagent for production according to claim 6, wherein the reagent comprises an expression inhibitor of the yeast 110315 gene and is for use in the production of a yeast with improved oil and/or fat production ability.

9. An oil and/or fat producing yeast, which is in a state where expression of a yeast 110315 gene is reduced.

10. A composition for producing an oil and/or fat, comprising the oil and/or fat producing yeast according to claim 9.

11. A production method of an oil and/or fat, the method comprising recovering an oil and/or fat from at least one selected from the group consisting of a culture of the oil and/or fat producing yeast according to claim 9 and a composition for producing an oil and/or fat comprising said oil and/or fat producing yeast.
